(19) United States
(12) Patent Application Publication
(10) Pub. No.: US 2012/0157532 A1
(43) Pub. Date: Jun. 21, 2012

(54) Method Of Treating Or Preventing A Convulsive Disorder In A Patient In Need Thereof

(76) Inventors: Serge Picaud, Paris (FR); Jose-Alain Sahel, Paris (FR); Manuel Simonutti, Paris (FR); Firas Jammoul, Lattaquie (SY)

(21) Appl. No.: 13/265,349

(22) PCT Filed: Apr. 16, 2010
(86) PCT No.: PCT/EP2010/055053

§ 371 Date: Mar. 7, 2012

Related U.S. Application Data
(60) Provisional application No. 61/171,210, filed on Apr. 21, 2009.

(51) Int. Cl. A61K 31/197; C07C 229/30; A61P 25/08

(52) U.S. Cl. 514/561; 562/574

(57) ABSTRACT

The present invention relates to a method of treating or preventing a convulsive disorder in a patient in need thereof comprising administering said patient with a therapeutically effective amount of an active ingredient that induces a high level of extracellular GABA or increases GABA receptor activation once per day in the evening or at night.

### FIELD OF THE INVENTION

The invention generally relates to compositions of an active ingredient that induces a high level of extracellular GABA or increases GABA receptor activation for treating convulsive disorders and methods of treating convulsive disorders employing such compositions.

### BACKGROUND OF THE INVENTION

A deficiency of GABA in the brain has been implicated as one cause for convulsions. (Karlsson, A.; Funnum, F.; Malthe-Sorrensen, D.; Storm-Mathisen, J. Biochem Pharmacol 1974, 22, 3053-3061). To correct the deficiency of brain GABA and therefore stop convulsions, an important approach is to use an inhibitor of GABA-aminotransferase (GABA-AT) that is able to cross blood-brain barrier. (Nanavati, S. M.; Silverman, R. B. J. Med. Chem. 1989, 32, 2413-2421.). Inhibition of this enzyme increases the concentration of GABA in the brain, which has therapeutic applications in epilepsy as well as other neurological disorders. One of the most effective in vivo time-dependent inhibitors of GABA-AT is 4-amino-5-hexenoic acid, which is also termed gamma-vinyl GABA or vigabatrin, an anticonvulsant drug marketed almost all over the world.

Vigabatrin is an anti-epileptic drug blocking the GABA-transaminase. In patients, the plasma VGB concentration peaks within an hour of oral administration to then decrease to half of the peak concentration within six to eight hours (Rey et al., 1992). By contrast, the VGB-elicited irreversible block of the GABA-transaminase results in longer lasting effects on the GABA concentration because the reversibility requires the synthesis of new GABA-transaminase molecules. In 1987, vigabatrin was found to induce an irreversible constriction of the visual field (Eke et al., 1997; Krauss et al., 1998). Recently, it was demonstrated that the retinal toxicity of vigabatrin is due to an increase in sensitivity to phototoxicity (Jammoul et al., 2009).

Despite these irreversible visual effects, Vigabatrin remains in infantile spasms the only alternative to adrenocorticotrophic hormone (ACTH) or steroid therapy (Ben-Menachem E. et al. 2008; Chiron C. et al. 1997; Lux Al. et al. 2005; Dulac 0. et al. 2008; Snead OC. Et al. 1983; Hrachovy RA. et al. 1994; Baram TZ. et al. 1996). It is also prescribed as a third-line drug for other refractory epilepsies in Europe (Ben-Menachem E. et al. 2008). Furthermore, it is being evaluated for treatment of heroin, cocaine and methamphetamine addictions (Gerasimov M R. et al. 1999; Stromberg M F. et al. 2001).

### SUMMARY OF THE INVENTION

The present invention relates to a method of treating or preventing a convulsive disorder in a patient in need thereof comprising administering said patient with a therapeutically effective amount of an active ingredient that induces a high level of extracellular GABA or increases GABA receptor activation. The method proposes to administer the ingredient only once per day and to achieve this administration in the evening or at night to limit the ingredient's phototoxic consequences.

Therefore the present invention relates to a method of treating or preventing a convulsive disorder in a patient in need thereof comprising administering said patient with a therapeutically effective amount of an active ingredient that induces a high level of extracellular GABA or increases GABA receptor activation once per day in the evening or at night.

### DETAILED DESCRIPTION OF THE INVENTION

The present invention relates to:
i. a method of treating or preventing a convulsive disorder in a patient in need thereof comprising administering said patient with a therapeutically effective amount of an active ingredient that induces a high level of extracellular GABA or increases GABA receptor activation once per day in the evening or at night; and to:
ii. an active ingredient that induces a high level of extracellular GABA or increases GABA receptor activation for use in the treatment or prevention by administration once per day in the evening or at night, e.g. at bed time or prior to sleep, of a convulsive disorder.

As used herein, the term “active ingredient that induce a high level of extracellular GABA” refers to a compound natural or not that has the capability to increase the concentration of GABA in the brain, which has therapeutic applications in convulsive disorder. The term “active ingredient that increases GABA receptor activation” refers to a compound natural or not that has the capability to activate GABA receptor.

Active ingredients that induce a high level of extracellular GABA or increases GABA receptor activation encompass GABA-aminotransferase inhibitors, GABA transporter inhibitors, Glutamate decarboxylase activators and GABA receptor agonists or modulators. GABA-aminotransferase, may also be termed GABA-transaminase or 4-aminobutyrate transaminase (EC 2.6.1.19). Glutamate decarboxylase is classified as EC 4.1.1.15.

As intended herein, GABA-aminotransferase inhibitors encompass 4-amino-5-hexenoic acid (vigabatrin), valproate, (1 R,3S,4S)-3-amino-4-fluorocyclopentane-1 -carboxylic acid, (1 R,4S)-4-amino-2-cyclopentene-1 -carboxylic acid, (1 S,4R)-4-amino-2-cyclopentene-1-carboxylic acid, (4R)-4-amino-1-cyclopentene-1-carboxylic acid, (4S)-4-amino-1-cyclopentene-1-carboxylic acid, (S)-4-amino-4,5-dihydro-2-thiophenecarboxylic acid, 1 H-tetrazole-5-(alpha-vinyl-propanamine), 2,4-Diaminobutanoate, 2-Oxoadipic acid, 2-Oxoglutarate, 2-Thiouracil, 3-Chloro-4-aminobutanoate, 3-Mercaptopropionic acid, 3-Methyl-2-benzothiazolone hydrazone hydrochloride, 3-Phenyl-4-aminobutanoate, 4-ethynyl-4-aminobutanoate, 5-Diazouracil, 5-Fluorouracil, Aminooxyacetate, beta-Alanine, Cycloserine and D-Cycloserine. As intended herein; glutamate decarboxylase activators encompass 2-Oxoglutarate, 3-Mercaptopropionic acid, Aminooxyacetic acid and Glutarate.

The GABA transporter inhibitor may consist of tiagabine. The GABA receptors agonists and modulators: may be selected from the group consisting of topiramate, felbamate, tramadol, Oxcarbazepine, Carbamazepine, eszopiclone, zopiclone, baclofen, gamma-Hydroxybutyric acid, imidazopyridines like zaleplon, Zolpidem, zopiclone phenytoin, propofol, phenytoin, benzodiazepines and barbiturates.

Benzodiazepines may be selected from the group consisting of clobazam, Alprazolam (Xanax®), Bromazepam (Lexomil®), Diazepam (Valium®), Lorazepam (Ativan®), Clonazepam (Klonopin®), Temazepam (Restoril®), Oxazepam (Serax®), Flunitrazepam (Rohypnol®), Triazolam (Halcion®), Chlordiazepoxide (Librium®), Flurazepam (Dalmane®), Estazolam (ProSom®), and Nitrazepam (Mogadon®).

Barbiturates may be selected from the group consisting of primidone and phenobarbitone, pentobarbital, midazolam, phenytoin, secobarbital and amobarbital butabarbital barbital, phenobarbital, butalbital, cyclobarbital, allobarbital, methylphenobarbital, and vinylbital.

In a preferred embodiment, the active ingredient that induces a high level of extracellular GABA or increases GABA receptor activation is vigabatrin. The term “vigabatrin” refers to 4-amino-5-hexenoic acid that is commercially available under the name of SABRIL®. The term encompasses the racemic mixture of vigabatrin or the active isomer.

According to the invention, the term “patient in need thereof”, is intended for a human or a non-human mammal that shall be treated for a convulsive disorder. The patients in need of such treatments encompass those, either adult or child patients, which are susceptible to various convulsive disorders including primarily convulsive disorders. Convulsive disorders encompass epilepsy, tuberous sclerosis, infantile spasms as well as the convulsive disorders affecting patients undergoing a drug addiction, including a drug addiction to heroin or cocaine, and ethanol.

Generally speaking, a “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent; i.e., a carrier, or administration vehicle. Further, it is intended to mean an amount sufficient to reduce and most preferably prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluents; i.e., carrier, or additive.

In a particular embodiment, the active ingredient that induces a high level of extracellular GABA or increases GABA receptor activation is orally administered prior to sleep. In a particular embodiment, the active ingredient that induces a high level of extracellular GABA or increases GABA receptor activation is orally administered at bed time.

The method of the invention may further comprise comprising a step of administering, said patient with a therapeutically effective amount of a second active ingredient selected from the group consisting of taurine, a taurine precursor, a taurine metabolite, a taurine derivative, a taurine analog and a substance required for the taurine biosynthesis.

Said association was described in the International Patent Application WO2009/004082 for preventing or inhibiting the undesirable side-effects on retinal toxicity caused by an active ingredient that induces a high level of extracellular GABA or increases GABA receptor activation.

The term “taurine” refers to 2-am inoethanesulfonic acid.

As used herein, “taurine precursors” encompass substances that, when they are administered to a human or an animal, can be transformed, directly or indirectly, into taurine. Taurine precursors are selected from the group consisting of cysteine, cystathionine, homocysteine, S-adenosylhomocysteine, serine, N-acetyl-cysteine, glutathione, N-formylmethionine, S-adenosylmethionine, betaine and methionine.

As used herein, “taurine metabolites” encompass substances that are produced in vivo by transformation of taurine. Taurine metabolites are preferably selected from the group consisting of hypotaurine, thiotaurine, taurocholate.

As used herein, “taurine derivatives” encompass substances that are structurally close to taurine but possess at least one structural difference, such as one or more chemical changes, e.g. at least one replacement of an atom or a chemical group found in taurine by a distinct atom or a distinct chemical group. Taurine derivatives are preferably selected from different entities including the group consisting of acetylhomotaurinate, and piperidino-, benzamido-, phthalimido- or phenylsuccinylimido taurine derivatives. Such taurine derivatives are described notably by Kontro et al. (1983, Prog Clin Biol Res, Vol. 125: 211-220) and by Andersen et al. (2006, Journal of pharmaceutical Sciences, Vol. 73(n °1): 106-108). Derivatives include for instance taurolidine (4,4′-methylene-bis(tetrahydro-2H-1,2,4-thiadiazine-1,1-dioxide or taurolin), taurultam and taurinamide, chlorohydrate-N-isopropylamide-2-(1-phenylethyl)aminoethanesulfonic acid.

As used herein, “taurine analogs” encompass substances that are chemically distinct from taurine but which exert the same biological activity. Taurine analogs are preferably selected from the group consisting of (+/±)piperidine-3-sulfonic acid (PSA), 2-aminoethylphosphonic acid (AEP), (+/±)2-acetylaminocyclohexane sulfonic acid (ATAHS), 2-aminobenzenesulfonate (ANSA), hypotaurine, ±trans-2-aminocyclopentanesulfonic acid (TAPS) 8-tétrahydroquinoléine sulfonic acid (THQS), N-2-hydroxyethylpiperazine-N′-2-ethane sulphonic acid (HEPES), beta-alanine, glycine, guanidinoethylsulfate (GES), 3-acétamido-1-propanesulfonic acid (acamprosate).

As used herein, “substances required for taurine biosynthesis” encompass all substances that are involved in the in vivo taurine biosynthesis including enzymes and enzyme cofactors, thus including cysteine dioxygenase (EC 1.13.11), sulfinoalanine decarboxylase (EC 4.1.1.29) and cofactors thereof. Substances required for taurine biosynthesis are preferably selected from the group consisting of vitamin B6 (or pyridoxal-5′-phosphate), vitamin B12 (cobalamin), folic acid, riboflavin, pyridoxine, niacin, thiamine (thiamine pyrophosphate) and pantothenic acid.

Taurine precursors, taurine metabolites, taurine derivatives, taurine analogs and substances required for the taurine biosynthesis may be collectively termed “taurine-like substances”.

Said second active ingredient may be administered before, concomitantly or after the administration of the active ingredient that induces a high level of extracellular GABA or increases GABA receptor activation. For example, the second active ingredient may be administered in the evening or at night, preferably before to sleep. The second active ingredient may also be administered in the morning, preferably when the patient wakes up. Preferably, the second active ingredient is administered to said patient in the morning following the evening or night when the first active ingredient is administered to said patient.

The invention further pertains to a combination of (or a kit comprising):
an active ingredient that induces a high level of extracellular GABA or increases GABA receptor activation; and
a second active ingredient selected from the group consisting of taurine, a taurine precursor, a taurine metabolite, a taurine derivative, a taurine analog and a substance required for the taurine biosynthesis,
   for simultaneous or sequential use in the treatment or prevention of a convulsive disorder, wherein the active ingredient that induces a high level of extracellular GABA or increases GABA receptor activation is administered once per day in the evening or at night, e.g. at bed time or prior to sleep. The second active ingredient may for example be administered as described in the above paragraph.

The present invention also relates to a pharmaceutical composition that comprises the active ingredient that induces a high level of extracellular GABA or increases GABA receptor activation in combination or not with the second active ingredient as above described.

The pharmaceutical compositions according to the invention are suitable for treating various convulsive disorders including primarily convulsive disorders. Convulsive disorders encompass epilepsy, tuberous sclerosis, infantile spasms as well as the convulsive disorders affecting patients undergoing a drug addiction, including a drug addiction to heroin or cocaine, ethanol.

Thus, a pharmaceutical composition according to the invention consists primarily of an anti-convulsive pharmaceutical composition.

Typically, the pharmaceutical composition of the invention is adapted so that the dosage form used allows the administration of an amount of the active ingredient that induces a high level of extracellular GABA or increases GABA receptor activation (e.g. vigabatrin) ranging between 10 μg and 10 grams per day, preferably between 100 μg and 5 grams, including between 1 mg and 1 gram, for a human adult patient having a mean weight of 80 kilos. Lower amounts of the active ingredient may be used, especially when the active ingredient is not under the racemic form but instead under the form of its active isomer, which lower amounts are typically half the amount of the racemic form which would have been conventionally used.

In another particular embodiment, the amount of the second active ingredient, i.e. taurine or a taurine-like substance, is adapted so that the said pharmaceutical composition is adapted so that the dosage form used allows the administration of an amount of taurine or of the taurine-like substance ranging from 10 μg to 10 grams per day for a human adult patient having a mean weight of 80 kilos.

In a particular embodiment, the active ingredient(s) is (are) used in combination with one or more pharmaceutically or physiologically acceptable excipients.

Generally, a pharmaceutical composition according to the invention, irrespective of whether the said composition (i) comprises only one or more substances selected from the ingredient that induces a high level of extracellular GABA or increases GABA receptor activation or (ii) comprises a combination of a first active ingredient that induces a high level of extracellular GABA or increases GABA receptor activation and a second active ingredient selected from taurine and taurine-like substances, comprises the one or more active ingredients in an amount ranging from 0.1% to 99.9% by weight, and usually from 1% to 90% by weight, based on the total weight of the said pharmaceutical composition.

Generally, a pharmaceutical composition according to the invention comprises an amount of excipient(s) that ranges from 0.1% to 99.9% by weight, and usually from 10% to 99% by weight, based on the total weight of the said pharmaceutical composition.

By “physiologically acceptable excipient or carrier” is meant solid or liquid filler, diluents or substance which may be safely used in systemic or topical administration. Depending on the particular route of administration, a variety of pharmaceutically acceptable carriers well known in the art include solid or liquid fillers, diluents, hydrotropes, surface active agents, and encapsulating substances.

Pharmaceutically acceptable carriers for systemic administration that may be incorporated in the composition of the invention include sugar, starches, cellulose, vegetable oils, buffers, polyols and alginic acid. Specific pharmaceutically acceptable carriers are described in the following documents, all incorporated herein by reference: U.S. Pat. No. 4,401,663, Buckwalter et al. issued Aug. 30, 1983; European Patent Application No. 089710, LaHann et al. published Sep. 28, 1983; and European Patent Application No. 0068592, Buckwalter et al. published Jan. 5, 1983. Preferred carriers for parenteral administration include propylene glycol, pyrrolidone, ethyl oleate, aqueous ethanol, and combinations thereof.

Representative carriers include acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%.

For formulating a pharmaceutical composition according to the invention, the one skilled in the art will advantageously refer to the last edition of the European pharmacopoeia or of the United States pharmacopoeia.

Preferably, the one skilled in the art will refer to the fifth edition “2005” of the European Pharmacopoeia, or also to the edition USP 28-NF23 of the United States Pharmacopoeia.

A further object of the invention relates to an active ingredient that induces a high level of extracellular GABA or increases GABA receptor activation for use in the treatment of a convulsive disorder wherein said active ingredient is administered once per day in the evening or at night.

The invention will be further illustrated by the following figures and examples. However, these examples and figures should not be interpreted in any way as limiting the scope of the present invention.

### FIGURES

FIG. 1: The daytime dependence of the vigabatrin-induced retinal toxicity. (A) Quantification of photopic ERG amplitudes in control animals and vigabatrin-treated rats injected either in the morning (VGB AM) or in the evening (VGB PM) for 65 days. (B) Lengths of retinal areas with displaced photoreceptor nuclei in control animals and vigabatrin-treated rats injected either in the morning (VGB AM) or in the evening (VGB PM). Photoreceptor nuclei were stained by DAPI and viewed under UV illumination. (C) Lengths of retinal areas with increased GFAP staining in the outer retina in control animals and vigabatrin-treated rats injected either in the morning (VGB AM) or in the evening (VGB PM). Values are indicated as mean with s.e.m. (control, n=5; VGB AM, n=10; VGB PM, n=10, Statistical significance **p<0.001, ∞p<0.005, *p<0.01, °p<0.05).

### EXAMPLE

### Material & Methods

Animal treatments: As described previously (Duboc et al., 2004), Wistar rats Rj Wi IOPS Han were purchased from Janvier (Le Genest-St-lsle, France) at between six and seven weeks of age. VGB dissolved in 0.9% NaCl was administered at 40 mg (125 mg/ml, 0.32 ml) to rats by daily intraperitoneal injection for 65 days. These daily doses (rats: 200 mg/kg) are in-line with those described for the treatment of epilepsy in animals (Andre et al., 2001) or in humans (adult patients: 1-6 mg/kg; children: 50-75 mg/kg; or infants: 100-150 mg/kg) (Aicardi et al., 1996; Chiron et al., 1997; Lux et al., 2004). Light intensity in the animal cages ranged between 125 and 130 lux.

Electroretinogram (ERG): Photopic ERGs were recorded after the last VGB injection, as described previously (Duboc et al., 2004). Anesthesia was induced by intraperitoneal injection (0.8 to 1.2 ml/kg) of a solution containing ketamine (40 mg/ml) and xylazine (4 mg/ml Rompum). Animals were light-adapted for 10 minutes with a background light of 25 cdm⁻². Light flashes were then applied on this background light; the light intensity of the flash was 25 cdsm⁻². Ten recordings were averaged with an interstimulus interval of 30 s.

Histology: Eye cups were fixed overnight at 4° C. in 4 % (wt/vol) paraformaldehyde in phosphate buffered saline (PBS; 0.01 M, pH 7.4). The tissue was cryoprotected in successive solutions of PBS containing 10%, 20% and 30% sucrose at 4° C., oriented along the dorso-ventral axis and embedded in OCT (Labonord, Villeneuve d'Ascq, France). Retinal sections (8-10 μm thickness) were permeabilised for five minutes in PBS containing 0.1% Triton X-100 (Sigma, St. Louis, Mo.), rinsed, and incubated in PBS containing 1% bovine serum albumin (Eurobio, Les-Ulis, France), 0.1% Tween 20 (Sigma), and 0.1% sodium azide (Merck, Fontenay-Sous-Bois, France) for two hours at room temperature. The primary antibody added to the solution was incubated for two hours at room temperature. Polyclonal antibodies were directed against rabbit GFAP (1/400, Dako, USA). Sections were rinsed and then incubated with the secondary antibody, goat anti-rabbit IgG conjugated to Alexa TM488 (1:500, Molecular Probes, Eugene, Oreg.) for two hours. The dye, diamidiphenyl-indole (DAPI), was added during the final incubation period. Sections were rinsed, mounted with Fluorsave reagent (Calbiochem, San Diego, Calif.) and viewed with a Leica microscope (LEICA DM 5000B) equipped with a Ropper scientific camera (Photometrics cool SNAP™ FX).

For quantification, vertical sections along the dorso-ventral axis were selected at the optic nerve. Following DAPI nuclear staining, the lengths of disorganised retinal areas were measured; GFAP immunostaining was used for detection and quantificationof retinal areas with reactive gliosis.

Statistical analysis: Statistical analysis of the results was performed by a one-way analysis of variance with the Student-Newman Keuls test (Sigmastat) for all measurements.

### Results

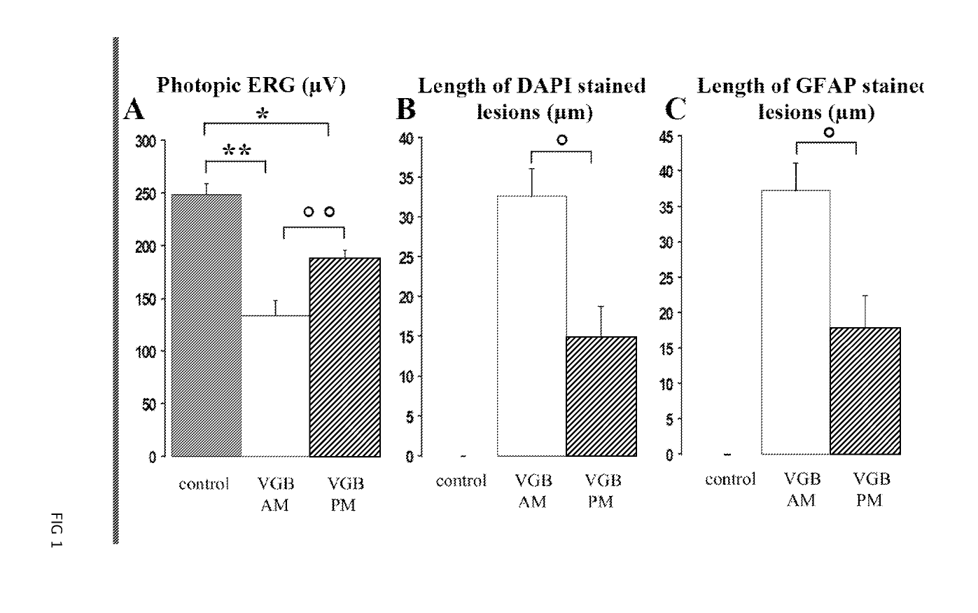

Animals were maintained in 12 h/12 h light/dark cycles. VGB was administered for 65 days either at the beginning (group VGB AM, n=10) or at the end of the light cycle (group VGB PM, n=10). As previously described (Duboc et al., 2004; Jammoul et al., 2009), photopic ERG measurements revealed a lower ERG amplitude in these two VGB-treated groups than in the control group (n=6) (FIG. 1A, **p<0.001, *p<0.01). However, the ERG amplitude decrease was less important in the group VGB PM injected at the end of the light cycle and the difference between the two VGB-treated groups was statistically significant (FIG. 1A, °°p<0.005). The level of disorganisatio n of the outer nuclear layer, previously reported (Butler et al., 1987; Duboc et al., 2004; Jammoul et al., 2009), was quantified on retinal sections. Animals treated in the evening (VGB PM) had smaller disorganised retinal areas than rats injected in the morning (VGB AM) (FIG. 1B, °p<0.05). Finally, the extent of retinal gliosis was revealed by GFAP immunolabelling. Both VGB-treated groups exhibited intense staining in the outer retina not seen in control animals. However, these GFAP-positive areas were less widely distributed in animals treated in the evening (VGB PM) than those treated in the morning (VGB AM) (FIG. 1C, °p<0.05). Therefore, all features of VGB-elicited retinal lesions were greater in VGB-treated animals injected in the morning than those administered VGB in the evening. These results suggest that the VGB retinal phototoxicity is related to the circulating VGB concentration during the day period. As the vigabatrin-induced irreversible inhibition of the GABA transaminase lasts for few days, VGB should be administered only in the evening to limit the VGB blood concentration during the day period and thus limit the occurrence of retinal lesions.

### REFERENCES

Throughout this application, various references describe the state of the art to which this invention pertains. The disclosures of these references are hereby incorporated by reference into the present disclosure.

Aicardi J, Mumford J P, Dumas C, Wood S. Vigabatrin as initial therapy for infantile spasms: a European retrospective survey. Sabril IS Investigator and Peer Review Groups. Epilepsia. 1996; 37:638-642

Andre V, Ferrandon A, Marescaux C, Nehlig A (2001) Vigabatrin protects against hippocampal damage but is not antiepileptogenic in the lithium-pilocarpine model of temporal lobe epilepsy. Epilepsy Res 47:99-117.

Baram T Z, Mitchell W G, Tournay A et al. High-dose corticotropin (ACTH) versus prednisone for infantile spasms: a prospective, randomized, blinded study. Pediatrics. 1996; 97:375-379

Ben-Menachem E, Dulac O, Chiron C. Vigabatrin. In: Epilepsy: a comprehensive textbook. Editors Jerome Engel Jr and Timothy A Pedley. Lippincott Williams & Wilkins, Philadelphia. 2008; Second edition: 1683-1693.

Buncic J R, Westall C A, Panton C M, Munn J R, MacKeen L D, Logan W J. Characteristic retinal atrophy with secondary “inverse” optic atrophy identifies vigabatrin toxicity in children. Ophtalmology 2004; 111:1935-42

Butler W H, Ford G P, Newberne J W. A study of the effects of vigabatrin on the central nervous system and retina of Sprague Dawley and Lister-Hooded rats. Toxicologic pathology. 1987; 15:143-148

Chiron C, Dumas C, Jambaque I, Mumford J, Dulac O (1997) Randomized trial comparing vigabatrin and hydrocortisone in infantile spasms due to tuberous sclerosis. Epilepsy Res 26:389-395.

Cubells J F, Blanchard J S, Makman M H. The effects of in vivo inactivation of GABA-transaminase and glutamic acid decarboxylase on levels of GABA in the rat retina. Brain research. 1987; 419:208-215

Duboc A, Hanoteau N, Simonutti M, Rudolf G, Nehlig A, Sahel J A, Picaud S (2004) Vigabatrin, the GABA-transaminase inhibitor, damages cone photoreceptors in rats. Ann Neurol 55:695-705.

Dulac O, Dalla Bernardina B, Chiron C. West syndrome. In: Epilepsy: a comprehensive textbook. Editors Jerome Engel Jr and Timothy A Pedley. Lippincott Williams & Wilkins, Philadelphia. 2008; Second edition: 2329-2335

Eke T, Talbot J F, Lawden M C (1997) Severe persistent visual field constriction associated with vigabatrin. Bmj 314:180-181.

Gerasimov M R, Dewey S L. Gamma-vinyl gamma-aminobutyric acid attenuates the synergistic elevations of nucleus accumbens dopamine produced by a cocaine/heroin (speedball) challenge. Eur J Pharmacol. 1999; 380:1-4

Halonen T, Lehtinen M, Pitkanen A, Ylinen A, Riekkinen P J (1988) Inhibitory and excitatory amino acids in CSF of patients suffering from complex partial seizures during chronic treatment with gamma-vinyl GABA (vigabatrin). Epilepsy Res 2:246-252.

Halonen T, Pitkanen A, Riekkinen P J (1990) Administration of vigabatrin (gamma-vinyl-gamma-aminobutyric acid) affects the levels of both inhibitory and excitatory amino acids in rat cerebrospinal fluid. J Neurochem 55:1870-1874.

Hilton E J, Cubbidge R P, Hosking S L et al. Patients treated with vigabatrin exhibit central visual function loss. Epilepsia. 2002; 43:1351-1359

Hrachovy R A, Frost J D, Jr., Glaze D G. High-dose, long-duration versus low-dose, short-duration corticotropin therapy for infantile spasms. The Journal of pediatrics. 1994; 124:803-806

Imaki H, Moretz R, Wisniewski H, Neuringer M, Sturman J (1987) Retinal degeneration in 3-month-old rhesus monkey infants fed a taurine-free human infant formula. J Neurosci Res 18:602-614.

Izumi Y, Ishikawa M, Benz A M et al. Acute vigabatrin retinotoxicity in albino rats depends on light but not GABA. Epilepsia. 2004; 45:1043-1048

Jammoul F, Wang Q, Nabbout R, Coriat C, Duboc A, Simonutti M, Dubus E, Craft C M, Ye W, Collins S D, Dulac O, Chiron C, Sahel J A, Picaud S (2009) Taurine deficiency is a cause of vigabatrin-induced retinal phototoxicity. Ann Neurol 65:98-107.

Johnson M A, Krauss G L, Miller N R et al. Visual function loss from vigabatrin: effect of stopping the drug. Neurology. 2000; 55:40-45

Krauss G L, Johnson M A, Miller N R (1998) Vigabatrin-associated retinal cone system dysfunction: electroretinogram and ophthalmologic findings. Neurology 50:614-618.

Lake N, Malik N (1987) Retinal morphology in rats treated with a taurine transport antagonist. Exp Eye Res 44:331-346.

Leon A, Levick W R, Sarossy M G (1995) Lesion topography and new histological features in feline taurine deficiency retinopathy. Exp Eye Res 61:731-741.

Lux A L, Edwards S W, Hancock E et al. The United Kingdom Infantile Spasms Study (UKISS) comparing hormone treatment with vigabatrin on developmental and epilepsy outcomes to age 14 months: a multicentre randomised trial. Lancet Neurol. 2005; 4:712-717

Lux A L, Edwards S W, Hancock E, Johnson A L, Kennedy C R, Newton R W, O'Callaghan F J, Verity C M, Osborne J P (2004) The United Kingdom Infantile Spasms Study comparing vigabatrin with prednisolone or tetracosactide at 14 days: a multicentre, randomised controlled trial. Lancet 364:1773-1778.

McDonagh J, Stephen L J, Dolan F M et al. Peripheral retinal dysfunction in patients taking vigabatrin. Neurology. 2003; 61:1690-1694

Miller N R, Johnson M A, Paul S R et al. Visual dysfunction in patients receiving vigabatrin: clinical and electrophysiologic findings. Neurology. 1999; 53:2082-2087

Neal M J, Cunningham J R, Shah M A, Yazulla S. Immunocytochemical evidence that vigabatrin in rats causes GABA accumulation in glial cells of the retina. Neuroscience letters. 1989; 98:29-32

Neal M J, Shah M A (1990) Development of tolerance to the effects of vigabatrin (gamma-vinyl-GABA) on GABA release from rat cerebral cortex, spinal cord and retina. Br J Pharmacol 100:324-328.

Pitkanen A, Matilainen R, Ruutiainen T, Lehtinen M, Riekkinen P (1988) Effect of vigabatrin (gamma-vinyl GABA) on amino acid levels in CSF of epileptic patients. J Neurol Neurosurg Psychiatry 51:1395-1400.

Rascher K, Servos G, Berthold G, Hartwig H G, Warskulat U, Heller-Stilb B, Haussinger D (2004) Light deprivation slows but does not prevent the loss of photoreceptors in taurine transporter knockout mice. Vision Res 44:2091-2100.

Ravindran J, Blumbergs P, Crompton J, Pietris G, Waddy H. Visual field loss associated with vigabatrin: pathological correlations. J Neurol. Neurosurg Psychiatry 2001; 70:787-9

Rey E, Pons G, Olive G (1992) Vigabatrin. Clinical pharmacokinetics. Clin Pharmacokinet 23:267-278.

Sills G J, Patsalos P N, Butler E et al. Visual field constriction: accumulation of vigabatrin but not tiagabine in the retina. Neurology. 2001; 57:196-200

Snead O C, 3rd, Benton J W, Myers G J. ACTH and prednisone in childhood seizure disorders. Neurology. 1983; 33:966-970

Stromberg M F, Mackler S A, Volpicelli J R et al. The effect of gamma-vinyl-GABA on the consumption of concurrently available oral cocaine and ethanol in the rat. Pharmacol Biochem Behay. 2001; 68:291-299

Wang Q P, Jammoul F, Duboc A et al. Treatment of epilepsy: the GABA-transaminase inhibitor, vigabatrin, induces neuronal plasticity in the mouse retina. Eur J Neurosci. 2008; 27:2177-2187

1. An active ingredient that induces a high level of extracellular GABA or increases GABA receptor activation formulated for use in the treatment or prevention by administration once per day in the evening or at night, of a convulsive disorder.

2. The active ingredient according to claim 1, wherein aid active ingredient that induces a high level of extracellular GABA or increases GABA receptor activation is administered prior to sleep.

3. The active ingredient according to claim 1, wherein said active ingredient that induces a high level of extracellular GABA or increases GABA receptor activation is selected from the group consisting of GABA-aminotransferase inhibitors, GABA transporter inhibitors, Glutamate decarboxylase activators and GABA receptor agonists or modulators.

4. The active ingredient according to claim 1, wherein said active ingredient that induces a high level of extracellular GABA or increases GABA receptor activation is selected from the group consisting of 4-amino-5-hexenoic acid (vigabatrin), valproate, (1 R,3S,4S)-3-amino-4-fluorocyclopentane-1-carboxylic acid, (1 R,4S)-4-amino-2-cyclopentene-1-carboxylic acid, (1 S ,4R)-4-amino-2-cyclopentene-1-carboxylic acid, (4 R)-4-amino-1-cyclopentene-1-carboxylic acid, (4S)-4-amino-1-cyclopentene-1 carboxylic acid, (S)-4-amino-4,5-dihydro-2-thiophenecarboxylic acid, 1 H-tetrazole-5-(alpha-vinyl-propanamine), 2,4-Diaminobutanoate, 2-Oxoadipic acid, 2-Oxoglutarate,2-Thiouracil, 3-Oh loro-4-aminobutanoate, 3-Mercaptopropionic acid, 3-Methyl-2-benzoth iazolone hydrazone hydrochloride, 3-Phenyl-4-aminobutanoate, 4-ethynyl-4-aminobutanoate, 5-Diazouracil, 5-Fluorouracil, Aminooxyacetate, beta-Alanine, Cycloserine and D-Cycloserine.

5. The active ingredient according to claim 1, wherein said active ingredient that induces a high level of extracellular GABA or increases GABA receptor activation is vigabatrin.

6. The active ingredient according to claim 5, wherein vigabatrin is administered as a racemic mixture or the active isomer.

7. A combination of:
the active ingredient according to claim 1; and
a second active ingredient selected from the group consisting of taurine, a taurine precursor, a taurine metabolite, a taurine derivative, a taurine analog and a substance required for the taurine biosynthesis, for simultaneous or sequential use in the treatment or prevention of a convulsive disorder.

8. The combination according to claim 7, wherein said second active ingredient is administered to said patient in the morning, following the evening or night when the first active ingredient is administered to said patient.

9. A method treating or preventing a convulsive disorder in a patient in need thereof, comprising the step of administering to said patient, once per day during evening or at night or prior to sleep, a therapeutically effective amount of an active ingredient which induces a high level of extracellular GABA or increases GABA receptor activation.

10. The method of claim 9 further comprising the step of administering to said patient a second active ingredient selected from the group consisting of taurine, a taurine precursor, a taurine metabolite, a taurine derivative, a taurine analog and a substance required for the taurine biosynthesis.

11. The method of claim 10 wherein both of said administering steps are performed sequentially.

12. The method of claim 10 wherein both of said administering steps are perfoimed simultaneously.

13. The method of claim 9 wherein said active ingredient is selected from the from the group consisting of GABA-aminotransferase inhibitors, GABA transporter inhibitors, Glutamate decarboxylase activators and GABA receptor agonists or modulators.

14. The method of claim 9 wherein said active ingredient is selected from the group consisting of 4-amino-5-hexenoic acid (vigabatrin), valproate, (1 R,3S,4S)-3-amino-4-fluorocyclopentane-1-carboxylic acid, (1 R,4S)-4-amino-2-cyclopentene-1-carboxylic acid, (1 S ,4R)-4-amino-2-cyclopentene-1-carboxylic acid, (4 R)-4-amino-1-cyclopentene-1-carboxylic acid, (4S)-4-amino-1-cyclopentene-1 carboxylic acid, (S)-4-amino-4,5-dihydro-2-thiophenecarboxylic acid, 1 H-tetrazole-5-(alpha-vinyl-propanamine), 2,4-Diaminobutanoate, 2-Oxoadipic acid, 2-Oxoglutarate,2-Thiouracil, 3-Oh loro-4-aminobutanoate, 3-Mercaptopropionic acid, 3-Methyl-2-benzoth iazolone hyd razone hydrochloride, 3-Phenyl-4-aminobutanoate, 4-ethynyl-4-aminobutanoate, 5-Diazouracil, 5-Fluorouracil, Aminooxyacetate, beta-Alanine, Cycloserine and D-Cycloserine.

15. The method of claim 9 wherein said active ingredient is vigabatrin.

16. The method of claim 15 wherein said vigabatrin is either a racemic mixture or an active isomer.
